(19) United States
(12) Patent Application Publication
(10) Pub. No.: US 2006/0159650 A1
(43) Pub. Date: Jul. 20, 2006

(54) Composition and method for covalently coupling an antithrombotic substance and a hydrophilic polymer

(75) Inventor: Larry A. Alegria, Bozeman, MT (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Bacterin, Inc., Belgrade, MT (US)

(21) Appl. No.: 11/036,065

(22) Filed: Jan. 14, 2005

(51) Int. Cl. A61K 31/785

(52) U.S. Cl. 424/78.3

(57) ABSTRACT

An antithrombotic composition includes a hydrophilic polymer backbone and an antithrombotic moiety covalently linked to the hydrophilic polymer backbone. The antithrombotic composition is formed by exposing a hydrophilic polymer including a hydroxyl group to an antithrombotic polysaccharide without having to first aminate the hydrophilic polymer.

### BACKGROUND OF THE INVENTION

The present invention relates generally to modified hydrophilic polymers. More specifically, the present invention relates to a composition including hydrophilic polymers with covalently attached moieties to improve biocompatibility of the composition.

Thrombosis is a major health problem that causes the deaths of several million people per year and imposes substantial economic costs. The development of medical devices that contact physiological fluids, particularly blood, is a rapidly developing area of medicine. When in contact with bodily fluids, the materials of medical devices can stimulate adverse host responses such as rapid thrombogenic action and serve as a focus for the formation of thrombi or blood clots. These adverse reactions can limit the type of materials suitable for use with medical devices and lead to the loss of function and subsequent removal of implanted medical devices.

Anticoagulant substances such as, for example, heparin have been used to coat medical devices. Coatings that include these anticoagulant substances have shown promise in combating the formation of thrombi as a response to materials that are foreign to the body.

### BRIEF SUMMARY OF THE INVENTION

The present invention includes various antithrombotic compositions and methods for producing the antithrombotic compositions. In the present invention, a hydrophilic polymer including a hydroxyl group and an antithrombotic macromolecule including an aldehyde group are covalently bonded through the aldehyde group of the antithrombotic macromolecule and the hydroxyl group of the hydrophilic polymer.

### BRIEF DESCRIPTION OF THE DRAWINGS

FIG. 1 is a diagram of an antithrombotic macromolecule covalently attached to a substrate through multipoint attachment.

FIG. 2 is a diagram of an antithrombotic macromolecule covalently attached to a substrate through an endpoint attachment.

FIG. 3 is a diagram of a reaction mechanism for covalently linking an aldehyde-activated antithrombotic macromolecule and a hydrophilic polymer including hydroxyl groups.

### DETAILED DESCRIPTION

The present invention includes a method for producing an antithrombotic composition to improve the biocompatibility of a substrate either coated with the antithrombotic composition or formed from the antithrombotic composition. The antithrombotic composition of the present invention includes a hydrophilic polymer backbone and an antithrombotic moiety attached to the polymer backbone through a covalent linkage. Using the method of the present invention, the antithrombotic moiety can be covalently attached to the polymer backbone without first providing an amino group on the polymer backbone.

The antithrombotic composition of the present invention is produced by reacting an aldehyde-activated antithrombotic macromolecule with a hydrophilic polymer including hydroxyl groups. The reaction may be carried out in the presence of activating agents or under activating conditions to assist in the formation of a covalent linkage between the aldehyde-activated antithrombotic macromolecule and the hydrophilic polymer. As used herein, the term “aldehyde-activated” is defined to include macromolecules having an aldehyde group located at a terminal end of the macromolecule, macromolecules having one or more aldehyde groups located at other locations of the macromolecule that are available to react with other molecules, or macromolecules having a plurality of aldehyde groups including at least one at a terminal end. The aldehyde on the aldehyde-activated antithrombotic macromolecule is believed to react with the hydroxyl groups of the hydrophilic polymer to covalently attach the antithrombotic macromolecule and the hydrophilic polymer through an endpoint attachment.

Anticoagulant substances can be attached to substrates through multiple types of attachment. Anticoagulant substances such as, for example, heparin can be attached to substrates through ionic attachments. Heparin, which is a polysaccharide that is highly negatively-charged, readily forms water insoluble ion complexes with substrates that include cationic compounds or moieties. However, due to the weak strength of the ionic bonds, ionically-bound heparin is susceptible to desorption or leaching from substrates. As such, ionically-bound heparin may not be suitable for applications requiring biocompatibility over an extended period of time.

Anticoagulant substances can also be attached to substrates through covalent linkages. Covalent bonds are stronger than ionic bonds and, as such, anticoagulant substances attached to substrates via covalent linkages are less prone to desorption or leaching from the substrates. Anticoagulant substances can be covalently attached to substrates through either “multipoint attachment” or “endpoint attachment.” Multipoint attachment occurs when an anticoagulant macromolecule randomly attaches to a substrate through a plurality of functional groups localized along the anticoagulant macromolecule, whereas end-point attachment occurs when the anticoagulant macromolecule attaches to a substrate through a functional group located on a terminal end of the anticoagulant macromolecule.

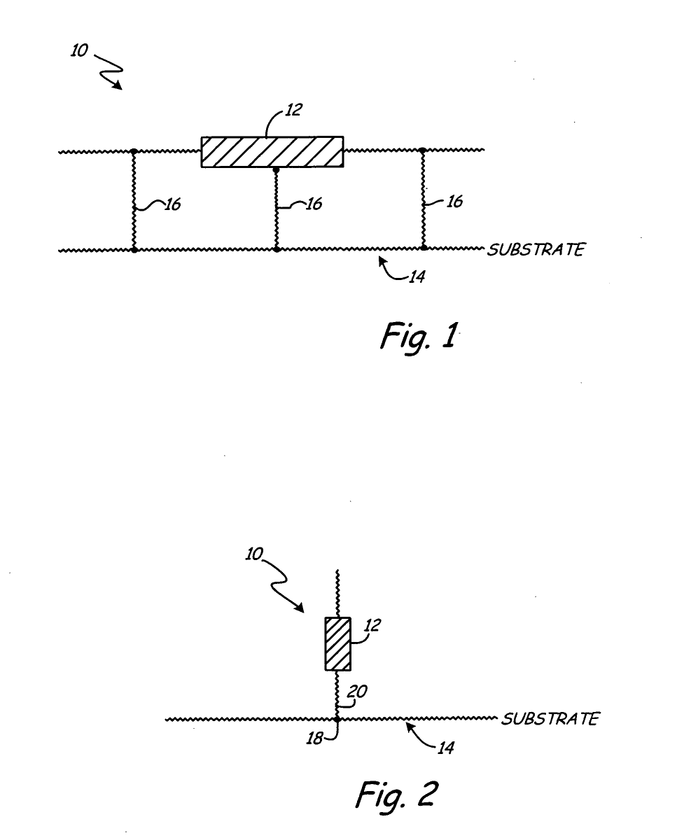

FIG. 1 shows a diagram illustrating antithrombotic macromolecule 10 having a discrete, biologically-active active site 12. Macromolecule 10 is attached to substrate 14 through multipoint attachment by a plurality of covalent linkages 16 formed at random locations along macromolecule 10. As shown in FIG. 1, these multiple attachments can affect the conformation of macromolecule 10 (including active site 12) and restrict or block access to active site 12, thereby affecting the antithrombotic (or anti-coagulation) properties of macromolecule 10.

FIG. 2 shows a diagram of antithrombotic macromolecule 10 of FIG. 1 covalently attached to substrate 14 by endpoint covalent linkage 18, which covalently links terminal end 20 of macromolecule 10 to substrate 14. Compared to the multipoint attachment of FIG. 1, the end-point attachment of FIG. 2 preserves the conformation of macromolecule 10, allows macromolecule 10 to extend outward from substrate 14, and maximizes access to active site 12. As such, endpoint attachment of macromolecule 10 to substrate 14 can facilitate the interaction of macromolecule 10 with other molecules and preserve the biological activity of macromolecule 10. Not surprisingly, end-point attachment is prevalent in nature with high molecular weight carbohydrates such as, for example, glycoproteins, glycolipids, proteoglycines, and lipopolysaccharides, which are immobilized by their reducing monosaccharide unit. This enables the high molecular weight carbohydrates to interact specifically with other molecules, such as, for example, plasma proteins, growth factors, antibodies, lectins and enzymes.

Methods for covalently attaching aldehyde-activated polysaccharides, such as heparin, to a substrate are known. These methods, however, entail first aminating the substrate to include amino groups, so the aldehyde-activated polysaccharides can be attached to the substrate through the amino groups. Once the substrate has been aminated, the aldehyde-activated polysaccharide is covalently attached through an aldehyde group to an amino group of the substrate in a reductive amination. A reducing agent such as cyanoborohydride is used for the reduction. These reducing agents are toxic and may pose a health risk if residual amounts remain associated with the polysaccharide and/or substrate. Unlike these methods, the method of the present invention does not require aminating the substrate and does not require using toxic reducing agents such as cyanoborohydride.

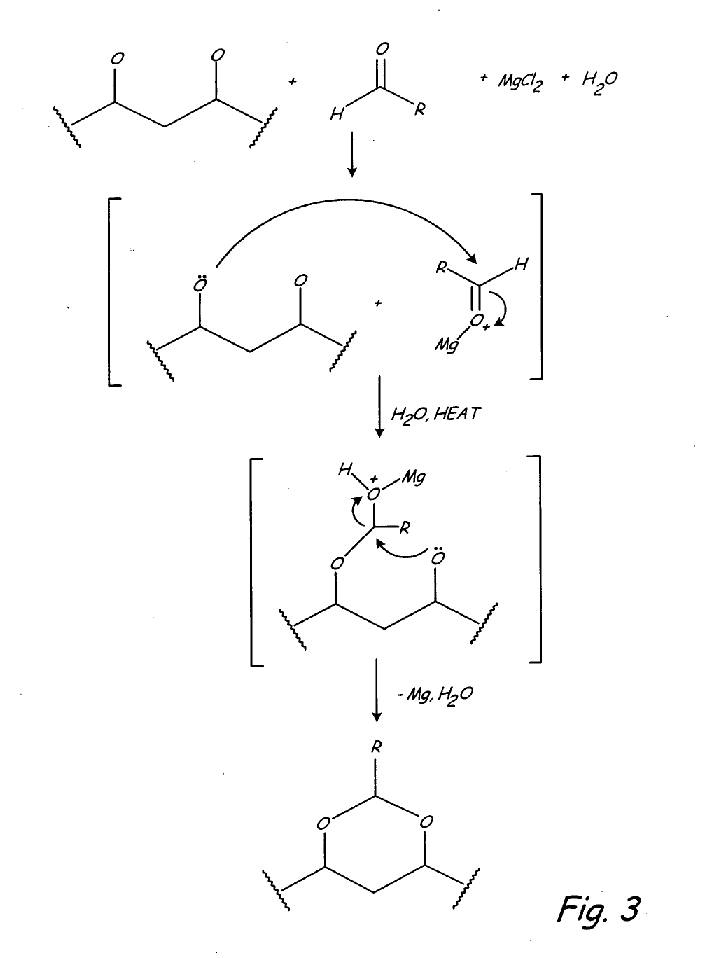

Based upon insights gained from the formation of poly(vinyl butryal) from poly(vinyl alcohol) and butyraldehyde, the formation of one embodiment of the antithrombotic composition of the present invention is believed to occur as shown in the reaction mechanism of FIG. 3. The reactants of FIG. 3 include an aldehyde-activated antithrombotic macromolecule R and a hydrophilic polymer molecule. FIG. 3 is a simplified reaction diagram and shows only a portion of the polymer backbone of the hydrophilic polymer molecule, which includes a pair of hydroxyl groups bonded to the polymer backbone in close proximity to one another.

As indicated in FIG. 3, the reaction is carried out in an aqueous solution including an activating agent (e.g., magnesium chloride). Magnesium activates the carbonyl carbon of the aldehyde group of macromolecule R and encourages the nucleophilic attack of the carbonyl-carbon by an oxygen from a hydroxyl group of the hydrophilic polymer molecule. An oxygen from a neighboring hydroxyl group of the same hydrophilic polymer molecule or a hydroxyl group of a different hydrophilic polymer molecule (not shown in FIG. 3) then reacts with the carbonyl carbon to form a covalent linkage in the form of an acetyl bridge. As such, the aldehyde-activated antithrombotic macromolecule may function as a bonding agent or a cross-linking agent for the hydrophilic polymer.

When both covalent linkages with macromolecule R are formed through hydroxyl groups of the same hydrophilic polymer molecule, the acetyl bridge constitutes a portion of a six-member ring structure. Similar to the ring structure included in poly(vinyl butyral), the six-member ring structure includes a pair of ether connections between the polymer backbone of the hydrophilic polymer molecule and the terminal carbon (i.e., the carbonyl carbon) of macromolecule R. It is believed that heating of the reaction solution encourages formation of the six-member ring structure.

Thus, pursuant to the reaction mechanism of FIG. 3, when macromolecule R is aldehyde-activated at a terminal end, macromolecule R is covalently attached to a single hydrophilic polymer molecule, or a pair of hydrophilic polymer molecules, through an endpoint covalent linkage. Likewise, when macromolecule R is aldehyde-activated at multiple locations (not shown in FIG. 3), macromolecule R is covalently attached to one or more hydrophilic polymer molecules through a plurality of covalent linkages.

Examples of suitable aldehyde-activated antithrombotic macromolecules for use in producing the antithrombotic composition of the present invention include heparin, chitin, chitosan, hyaluronic acid, dermatan sulfate, any other biocompatible or antithrombotic polysaccharide known in the art, any derivative of these, and any combination or copolymer of these in any proportion. The term “heparin,” as used herein, is defined to include any type of heparin, heparin derivative, heparin fraction (e.g., such as “high affinity” or “low affinity” heparin fractions), heparin-like substance, and any combination of these in any proportion. Examples of high affinity heparin include heparin capable of forming a complex with antithrombin and heparin including a pentasaccharide consisting of three D-glucosamine and two uronic acid residues, wherein the central D-glucosamine residue includes a 3-O-sulfate moiety.

In one embodiment of the present invention, the antithrombotic macromolecule is aldehyde-activated at a terminal end of the macromolecule. Deaminated heparin is one example of an antithrombotic macromolecule having an aldehyde at a terminal end. The synthesis, of deaminated heparin is described in U.S. Pat. No. 4,613,665, which is incorporated herein by reference. Deaminated heparin is formed by a diazotation reaction in which a six-member ring including an amine function is condensed to a five-member ring with a terminal aldehyde within the heparin structure (which results in the loss of the amine function in the ring).

Examples of suitable hydrophilic polymers for use in producing the antithrombotic composition of the present invention include polysaccharides with hydroxyl groups, polyalcohols, and any combination of these in any proportion. Examples of polysaccharides for use as the substrate material include chitin, chitosan, cellulose, hyaluronic acid, any derivative of these, and any combination or copolymer of these in any proportion. Although chemically distinct, the terms “chitin” and “chitosan” are used interchangeably herein.

In some embodiments of the present invention, the hydrophilic polymer includes poly(vinyl alcohol) (PVA). The percentage of hydrolysis for the PVA in these embodiments may be as low as about 60% percent hydrolysis and as high as about 100% percent hydrolysis. In one embodiment, the percentage of hydrolysis for the PVA is greater than about 99%. The molecular weight of the PVA may be as low as about 10,000 daltons and as high as about 186,000 daltons. In some embodiments, the molecular weight of the PVA may be as low as about 89,000 daltons and as high as about 98,000 daltons, while in still other embodiments the molecular weight of the PVA may be as low as about 124,000 daltons and as high as about 186,000 daltons.

The PVA in the antithrombotic composition of the present invention may comprise a single hydrolyzed form (in terms of percentage of hydrolysis), a mixture of a plurality of hydrolyzed forms; a single molecular weight form, a mixture of a plurality of molecular weight forms, or a mixture of any of these forms in any proportion.

The antithrombotic composition of the present invention may be applied to a substrate in the form of, for example, a coating (or layer). The coating can be applied to the substrate using any of the standard coating methods known in the art such as, for example, dipping, spraying, or rolling the substrate in a coating formulation including the hydrophilic polymer material. In one embodiment, the substrate is dipped for about one hour at a temperature of about 50° C. in a coating formulation. When the substrate to be coated contains a lumen, a vacuum or positive pressure may be applied during the application of the hydrophilic polymer to ensure that all parts of the substrate are exposed to the hydrophilic polymer.

Examples of suitable materials for substrates to be coated with hydrophilic polymer material include synthetic and naturally-occurring organic and inorganic polymers such as polyethylene, polypropylene, polyacrylates, polycarbonate, polyamides, polyurethane, polyvinylchloride (PVC), polyetherketone (PEEK), polytetrafluroethylene (PTFE), cellulose, silicone and rubber (polyisoprene), plastics, metals, glass, ceramics, any medical substrate material known in the art, derivatives of any of these, and any combination or copolymer of these in any proportion.

In some embodiments, the antithrombotic composition (or a component of the antithrombotic composition) is applied directly to a substrate material. Examples of substrate materials suitable for direct application include hydrophilic surfaces such as metals, glass, and cellulose. In other embodiments, the antithrombotic composition (or a component of the antithrombotic composition) is applied to pre-treated substrate material or to a primer coating on the substrate material. Examples of substrate materials that may require pre-treatment or priming include hydrophobic, surfaces such as silicone or PTFE.

The substrate material may be a medical device (or healthcare product), a portion of a medical device, a material used to construct a medical device, or other healthcare products. Examples of medical devices that can be coated with the antithrombotic composition of the present invention include catheters (such as, e.g., urological catheters and central venous catheters), guide wires, wound drains, orthopedic implants, dental implants, feeding tubes, tracheal tubes, sutures, and medication delivery products (e.g., needle-less connectors and/or IV products), and any other medical device or healthcare product that may contact bodily fluids.

Once the hydrophilic polymer has been applied to the substrate to form a coating, the coated substrate may be dried, either before and/or after contacting the coated substrate with a reaction mixture containing an aldehyde-activated antithrombotic macromolecule. Examples of suitable drying processes include air-drying, infrared radiation drying, convection or radiation drying (e.g., using a drying oven), forced air drying (e.g., using a heat gun) or any combination of these. In an exemplary embodiment, the coated substrate is dried overnight at room temperature.

In one embodiment, a partially dry or completely dry coating layer formed from the hydrophilic polymer is exposed to an aldehyde-activated antithrombotic macromolecule by immersing the coated substrate in a reaction mixture including the aldehyde-activated antithrombotic macromolecule. The coating may then be subjected to additional drying at room temperature for a pre-determined time. In some embodiments, the concentration of deamininated heparin in the reaction mixture may be as low as about 0.05 weight percent and as high as about 20 weight percent, while in an exemplary embodiment the concentration of the deamininated heparin in the reaction mixture is about 2.0 weight percent, based on the total weight of the reaction mixture.

In some embodiments of the present invention, an activating agent is included in the reaction mixture to assist in the covalent attachment of the antithrombotic macromolecule and the hydrophilic polymer. As used herein, the term “activating agent” is defined to include acids such as, for example, hydrochloric acid, sulfuric acid, citric acid, and acetic acid; Lewis acids such as, for example, magnesium-based Lewis acids (e.g., magnesium chloride), lithium-based Lewis acids, calcium-based Lewis acids, sodium-based Lewis acids, potassium-based Lewis acids, beryllium-based Lewis acids, strontium-based Lewis acids, manganese-based Lewis acids, aluminum-based Lewis acids, phosphorous-based Lewis acids, sulfur-based Lewis acids, copper-based Lewis acids, lead-based Lewis acids, and silver-based Lewis; and combinations of these in any proportion.

The concentration of the activating agent in the reaction mixture of the present invention may be as low as about 0.01 weight percent and as high as about 20 weight percent, based on the total eight of the reaction mixture. In some embodiments, the concentration of the activating agent in the reaction mixture is about 0.7 weight percent, based on the total weight of the reaction mixture.

Heat may also be applied to the reaction mixture to assist in the formation of a covalent linkage between the antithrombotic macromolecule and the hydrophilic polymer. In some embodiments, the reaction mixture of the present invention may be heated to a temperature as low as about 25° C. and as high as about 100° C. In an exemplary embodiment, the reaction mixture is heated to a temperature of about 50° C.

In some embodiments of the present invention, the antithrombotic composition may constitute a hydrogel. In addition, the antithrombotic composition, or a coating or medical device formed by the composition, may be loaded or impregnated with a biological agent, an antimicrobial, agent, an anti-cancer agent, or any combination of these in any proportion. The antithrombotic composition of the present invention may enhance the release kinetics of such agents when incorporated into the antithrombotic composition.

In some embodiments of the present invention, the antithrombotic composition includes a hydrophilic polymer, an antithrombotic moiety, and a linker covalently attaching the hydrophilic polymer and the antithrombotic moiety. This embodiment of the antithrombotic composition may be formed by covalently attaching a linker molecule to the hydrophilic polymer and then covalently attaching the antithrombotic macromolecule to the linker molecule. In one embodiment, the linker molecule is aldehyde-activated and forms a covalent linkage with the hydrophilic polymer through an aldehyde on the linker molecule and one or more hydroxyls on the hydrophilic polymer. In this embodiment, the linker has an additional functional group, such as for example a hydroxyl group, that is capable of reacting with the antithrombotic macromolecule to form a covalent linkage between the linker molecule and the antithrombotic macromolecule

### EXAMPLES

The present invention is more particularly described in the following examples that are intended as illustrations only, since numerous modifications and variations within the scope of the present invention will be apparent to those skilled in the art. Unless otherwise noted, all parts, percentages, and ratios reported in the following examples are on a weight basis, all reagents used in the examples were obtained, or are available, from commercial chemical suppliers or may be synthesized by conventional techniques.

Examples 1-7 illustrate one embodiment of a method of the present invention for producing a catheter having a hydrogel coating including PVA with covalently attached heparin.

### Example 1

### Preparation of PVA Coating Formulation

A PVA coating formulation was, prepared in an appropriate-sized vessel by adding PVA (5.0 g, molecular weight=89,000 to 98,000 daltons, 99% hydrolysis) to purified water and diluting to 100 mL. The coating formulation was then heated for one hour at about 75° C. to drive the PVA into solution. The resulting PVA coating formulation exhibited a clear white color and a smooth appearance.

### Example 2

### Preparation of Heparin Solution

A deaminated heparin solution was prepared by combining deaminated heparin (200 mg, commercially available from Celsus Laboratories, Inc.), magnesium chloride hexahydrate (500 mg, commercially available from Aldrich), and 10 mL of purified water. The deaminated heparin solution was then sonicated to make a clear solution.

### Example 3

### Substrate Pre-Treatment

A pretreated catheter was prepared as follows. Contaminants on the surface of the catheter, such as, for example, oil, mold, and release agents were removed by subjecting the catheter to a pressure of about 25 mTorr for a minimum of 3 minutes. An oxygen cleaning and etching step was performed by setting the power of a plasma apparatus at 495 Watts and increasing the pressure with oxygen to 120 mTorr. An allyl alcohol functionalization step was performed using a flow rate=0.17 ml per minute of alcohol for 8 minutes with 3% argon as a carrier gas at a pressure of about 50 mTorr. Alternatively, the allyl alcohol addition can also be done with 3% argon and 5% oxygen as the carrier gases. For further discussion relating to the pretreatment methodology, see U.S. Provisional Application No. 60/566,576 filed on Apr. 29, 2004 and entitled “Antimicrobial Coating for Inhibition of bacterial adhesion and biofilm formation,” which is incorporated herein by reference.

### Example 4

### PVA-Coating of a Catheter Substrate

The pre-treated catheter, of Example 3 was dipped for about 60 seconds into the PVA coating formulation of Example 1 at a temperature of about 38° C. The catheter was spun at 2 rpm during the immersion. The catheter was then mechanically withdrawn from the coating formulation at a withdrawal speed that varied from about 5 to 7 mm/second, while being spun at 5 rpm. The PVA-coated catheter was then dried overnight at room temperature.

### Example 5

### Bonding of Heparin to a PVA-Coated Catheter Substrate

Heparin was bonded to the PVA coated catheter of Example 4 using a dip process. The PVA-coated catheter of Example 4 was dipped for 1 hour at 50° C. into a tank containing the heparin solution of Example 2. The catheter was then withdrawn from the tank and dried over night at room temperature.

### Example 6

### Factor Xa Assay Results

A Factor Xa test was run to determine the heparin surface concentration and pharmaceutical activity of the heparin/PVA coating of Example 5. The coated catheter of Example 5 was placed in 10 ml of pH 7.4 phosphate buffered saline (PBS) and rocked for four days. The PBS was changed five times during the four days (two buffer changes the first day and one buffer change per day for each of the next three days). The coated catheter of Example 5 was then removed from the PBS and a 0.5 cm sample of the coated catheter of Example 5 was carefully cut and dried.

The catheter sample was placed in a 2 ml plastic vial and a Factor Xa test was performed using the Coatest® Heparin kit commercially available from DiaPharma. 200 ul of Tris buffer (pH 8.4) was added to the catheter sample followed by 20 ul of 1 IU/mL antithrombin III (comfmercially available from DiaPharma). The sample of the coated catheter of Example 5 (“coated catheter sample”) was vortexed and incubated at 37° C. for ten minutes. Next, 200 ul of Factor Xa (71 nkat) was added to the coated catheter sample, which was vortexed and incubated at 37° C. for 5 minutes. 200 ul of chromogenic substrate S-2222 was then added to the coated catheter sample. After mixing the coated catheter sample for 10 minutes at 37° C., absorbance of the chromophoric group at 405 nm was measured.

To assess the pharmaceutical activity of the coated catheter sample, a standard curve of absorbances at 405 nm was made using standards having respective concentrations of 0.01, 0.03, 0.05, and 0.07 Iu/mL of deaminated heparin (commercially available from Celsus Laboratories, Inc.). The standards and the coated catheter sample were processed side-by-side on the same day. The coated catheter sample had a pharmaceutical activity within the pharmaceutical activity range for the heparin standards. Using the standard curve, the coated, catheter sample was determined to have a surface heparin concentration of approximately 8 ug/cm². Thus, an appreciable amount of biologically-active heparin remained attached to the coated catheter sample after agitation and repeated washing over an extended period of time.

### Example 7

### Toluidine Blue Assay Results

A semi-quantitative toluidine blue assay was performed to determine the concentration of heparin at the surface of the coated catheter of Example 5. Positively charged toluidine blue dye ionically associates with negatively charged sulfonic and carboxylic groups of heparin, producing a chromophore that results in a violet color on the surface of a heparin-containing coating.

A two-centimeter sample of the coated catheter of Example 5 (“experimental sample”) was both washed with PBS and dried pursuant to the PBS washing and subsequent drying procedures described above for Example 6. A two-centimeter control sample was also prepared using a sample of the PVA-coated catheter of Example 4 washed and dried in the same manner as the experimental sample. The experimental sample and the control sample were then each immersed for about five minutes in a PBS solution containing toluidine blue (35 mg/ml). The experimental and control samples were then carefully washed with cold water and dried. The experimental sample exhibited a homogeneous violet color signifying the presence of heparin while the control sample remained a clear white color.

The experimental and the control sample were then each immersed for about ten minutes in a room temperature solution of 1.4 ml of 1% Sodium Dodecyl Sulfate (SDS). The absorbances of the SDS solutions at 600 nm for the experimental and the control sample were then measured. The control sample exhibited an absorbance of 0.002 and the experimental sample exhibited an absorbance of 0.054. These results indicated the semi-quantitative presence of heparin on the surface of the experimental sample through the uptake of toluidine blue stain. Thus, an appreciable amount of heparin remained attached to the experimental sample after agitation and repeated washing over an extended period of time.

The above Factor Xa and toluidine blue assay results both indicate that an appreciable amount of heparin remained attached to the surface of the PVA coating of Example 5 even after subjecting the coating,to agitation and repeated washing over an extended period of time. In addition, the Factor Xa results indicated that the attached heparin was biologically-active. As such, these results indicate that the heparin attached to the surface of the PVA coating through an endpoint covalent linkage.

Thus, as described above, the method of the present invention provides an efficient process for covalently linking an aldehyde-activated antithrombotic macromolecule and a hydrophilic polymer. Unlike previous methods, the method of the present invention does not require an amination step and does not require using toxic reducing agents.

Although the present invention has been described with reference to preferred embodiments, workers skilled in the art will recognize that changes may be made in form and detail without departing from the spirit and scope of the invention.

1. A method for treating a substrate to resist thrombosis, the method comprising: providing the substrate having at a surface a hydrophilic polymer including a hydroxyl group; and covalently bonding an antithrombotic macromolecule to the hydrophilic polymer through a terminal end of the antithrombotic macromolecule and the hydroxyl group of the hydrophilic polymer.

2. The method of claim 1 and further comprising:
coating the surface of the substrate with the hydrophilic polymer to form a hydrogel layer on the surface.

3. The method of claim 1 and further comprising:
forming the substrate from the hydrophilic polymer.

4. The method of claim 1, and further comprising:
exposing the antithrombotic macromolecule to an activating agent.

5. The method of claim 1, wherein the hydrophilic polymer is exposed to the antithrombotic macromolecule at a temperature of greater than about 25° C. and less than about 100° C.

6. The method of claim 1, wherein the hydroxyl group is located on the backbone of the hydrophilic polymer.

7. The method of claim 6, wherein the covalent bond is formed through an aldehyde group located on the terminal end of the antithrombotic macromolecule.

8. The method of claim 1, wherein the hydrophilic polymer comprises a polyalcohol.

9. The method of claim 8, wherein the polyalcohol comprises a poly(vinyl alcohol).

10. The method of claim 1, wherein the hydrophilic polymer comprises a polysaccharide.

11. The method of claim 1, wherein the antithrombotic macromolecule comprises an antithrombotic polysaccharide.

12. The method of claim 11, wherein the antithrombotic polysaccharide is selected from the group consisting of heparin, chitosan, hyaluronic acid, dermatan sulfate, and any combination or copolymer of these.

13. A method for producing an antithrombotic composition, the method comprising covalently attaching heparin to a hydroxyl group of a hydrophilic polymer.

14. The method of claim 13, wherein the covalently-attached heparin is biologically active.

15. The method of claim 13, wherein the heparin comprises an aldehyde-activated heparin.

16. The method of claim 15, wherein an aldehyde group located at a terminal end of the aldehyde-activated heparin is reacted with the hydroxyl group of the hydrophilic polymer to covalently attach the heparin and the hydrophilic polymer.

17. The method of claim 13, wherein a terminal end of the heparin is covalently attached to a pair of hydroxyl groups of the hydrophilic polymer.

18. The method of claim 13, wherein the hydrophilic polymer comprises a poly(vinyl alcohol).

19. The method of claim 13, wherein the heparin is covalently attached to the hydrophilic polymer in the presence of an activating agent.

20. The method of claim 13, wherein the heparin and the hydrophilic polymer are covalently attached at a temperature of greater than about 25° C. and less than about 100° C.

21. A method for treating a medical device to improve biocompatibility, the method comprising:
providing, on a surface of the medical device, a hydrophilic polymer including a hydroxyl group; and
exposing the hydrophilic polymer to an antithrombotic polysaccharide, without previously aminating the hydrophilic polymer to include an amino group, to covalently link the polysaccharide and the hydrophilic polymer.

22. The method of claim 21, and further comprising:
exposing the hydrophilic polymer and the polysaccharide to activating conditions to assist in the covalent linking of the polysaccharide and the hydrophilic polymer.

23. The method of claim 22, wherein the activating conditions comprise exposure to an activating agent.

24. The method of claim 23, wherein the activating conditions comprise a reaction temperature of greater than about 25° C. and less than about 100° C.

25. The method of claim 21, wherein the hydrophilic polymer comprises poly(vinyl alcohol).

26. The method of claim 21, wherein the polysaccharide is selected from the group consisting of heparin, chitosan, hyaluronic acid, dermatan sulfate, and any combination or copolymer of these.

27. A method for treating a medical device to improve biocompatibility, the method consisting of:
providing, on a surface of the medical device, a hydrophilic polymer including a hydroxyl group; and
exposing the hydrophilic polymer to an antithrombotic polysaccharide to covalently attach a terminal end of the polysaccharide to the hydrophilic polymer.

28. The method of claim 27, wherein the polysaccharide is covalently attached to the hydrophilic polymer at a surface of a layer formed by the hydrophilic polymer.

29. The method of claim 27, and further comprising:
exposing the hydrophilic polymer and the polysaccharide to activating conditions to assist in the covalent attachment of the polysaccharide and hydrophilic polymer.

30. The method of claim 29, wherein the activating conditions comprise exposure to an activating agent.

31. The method of claim 29, wherein the activating conditions comprise a reaction temperature of greater than about 25° C. and less than about 100° C.

32. The method of claim 27, wherein the hydrophilic polymer comprises poly(vinyl alcohol).

33. The method of claim 27, wherein the polysaccharide is selected from the group consisting of heparin, chitosan, hyaluronic acid, dermatan sulfate and any combination or copolymer of these.

34. A composition comprising a hydrophilic polymer backbone with an antithrombotic moiety covalently linked to the hydrophilic polymer backbone through a six-member ring structure.

35. The composition of claim 34, wherein the six-member ring structure includes a pair of ether connections between members of the ring.

36. The composition of claim 34, wherein the antithrombotic moiety comprises a polysaccharide.

37. The composition of claim 36, wherein the polysaccharide is selected from the group consisting of heparin, chitosan, hyaluronic acid, dermatan sulfate, and an combination or copolymer of these.

38. An antithrombotic composition comprising:
a poly(vinyl alcohol); and
a heparin covalently attached to the poly(vinyl alcohol) through an ether connection at a terminal end of the heparin.

39. The composition of claim 38, wherein the heparin is covalently attached to the poly(vinyl alcohol) through a pair of ether connections.

40. The composition of claim 39, wherein the pair of ether connections are included in a six-member ring that covalently attaches the terminal end of the heparin to the poly(vinyl alcohol).
